(19) United States
(12) Patent Application Publication
(10) Pub. No.: US 2010/0004623 A1
(43) Pub. Date: Jan. 7, 2010

(54) Method for Treatment of Complications Associated with Arteriovenous Grafts and Fistulas Using Electroporation

(75) Inventors: William C. Hamilton, JR., Queensbury, NY (US); James J. Mitchell, Ballston Spa, NY (US)

Correspondence Address: AFS / ANGIODYNAMICS, 666 THIRD AVENUE, FLOOR 10, NEW YORK, NY, 10017, US

(73) Assignee: AngioDynamics, Inc.

(21) Appl. No.: 12/561,064

(22) Filed: Sep. 16, 2009

Related U.S. Application Data
(63) Continuation-in-part of application No. 12/413,332, filed on Mar. 27, 2009.
Application No. 12/413,332 is a continuation-in-part of application No. 12/413,357, filed on Mar. 27, 2009.
(60) Provisional application No. 61/156,368, filed on Feb. 27, 2009.
(60) Provisional application No. 61/040,110, filed on Mar. 27, 2008.
(60) Provisional application No. 61/156,368, filed on Feb. 27, 2009.
(60) Provisional application No. 61/040,110, filed on Mar. 27, 2008.

(51) Int. Cl. A61N 1/30; A61B 18/18

(52) U.S. Cl. 604/501; 606/41

(57) ABSTRACT

Electroporation devices and methods for use in the treatment of complications, such as thrombosis, stenotic segments, or infections, associated with an arteriovenous graft or fistula are provided. The devices include at least two electrodes. The electrodes are adapted to be positioned near the target zone of complication for applying electrical pulses and thereby causing electroporation. In a preferred embodiment, the electroporation pulses are sufficient to subject substantially all cells within the target zone to irreversible electroporation without creating a thermally damaging effect.

### CROSS REFERENCE TO RELATED APPLICATION

This application is a continuation-in-part of prior U.S. application Ser. No. 12/413,332, filed Mar. 27, 2009, entitled “IRREVERSIBLE ELECTROPORATION DEVICE AND METHOD FOR ATTENUATING NEOINTIMAL”, which claims the benefit of U.S. Provisional Application Nos. 61/156,368, filed Feb. 27, 2009, and 61/040,110, filed Mar. 28, 2008, all of which are fully incorporated by reference herein.

This application is also a continuation-in-part of prior U.S. application Ser. No. 12/413,357, filed Mar. 27, 2009, entitled “BALLOON CATHETER METHOD FOR REDUCING RESTENOSIS VIA IRREVERSIBLE ELECTROPORATION”, which claims the benefit of U.S. Provisional Application Nos. 61/156,368, filed Feb. 27, 2009, and 61/040,110, filed Mar. 28, 2008, all of which are fully incorporated by reference herein.

### FIELD OF THE INVENTION

The present invention relates to a medical device and method for the treatment of complications arising from long term venous access devices. More particularly, the present invention relates to an electroporation device and method for treatment of thrombosis, infection and stenoses associated with arteriovenous grafts and fistulas.

### BACKGROUND OF THE INVENTION

Hemodialysis is the most common method of treating advanced and permanent kidney failure. Hemodialysis is the process by which blood is withdrawn from the patient's body and pumped through a dialysis machine that removes wastes and excess fluids from the blood before it is returned to the patient. There are three main methods for accessing a patient's blood during dialysis treatments: a primary arteriovenous (A/V) fistula, an A/V graft, or a central venous catheter. An A/V fistula is a surgical connection between an artery and vein through anastomosis, usually involving the radial artery and the cephalic vein. An A/V fistula must “mature” for two to four months before it can be used for hemodialysis. An A/V graft, which is created by joining an artificial vessel (e.g. a plastic tube) in a U-shape to both an artery and a vein, may be ready for use after several weeks and in some cases after only 48 hours. A central venous catheter may be used immediately but is not the access option recommended by many physicians, unless no other access routes are available.

As used herein, “graft” is inclusive of an A/V graft and A/V fistula. Regardless of which type of graft is used, complications occur in many patients soon after the arteriovenous graft is implanted. Complications include graft thrombosis, infection, stenosis of the graft-vein anastomosis and pseudo-aneurysms. Thrombosis, or blood clot formation, is the most common cause of graft failure. Various techniques known in the art are used to clear any in-graft thrombus. These techniques include surgical thrombectomy, graft replacement or percutaneous endovascular thrombolysis. Percutaneous thrombolysis is the least invasive option and has rapidly become the preferred method of treatment at most institutions. It can be accomplished using mechanical thrombectomy devices which macerate the clot mass or by using a thrombolytic agent to dissolve the clot. For example, tissue plasminogen activators are often introduced into a clotted graft via an infusion catheter or needle. Both the mechanical and pharmological treatments of grafts are time-consuming, invasive, expensive and often do not totally eliminate the thrombus.

Graft thrombosis usually results from venous flow obstruction or stenosis. Venous stenosis is present in over eighty-five percent of clotted grafts. The underlying venous anastamotic stenosis must be corrected in order to avoid recurrence of the thrombus. The location of the stenosis is most commonly found at the graft-to-vein anastomosis. The narrowing at this area causes a slow down or obstruction of blood flow resulting in the formation of thrombus within the graft. The underlying venous anastomic stenosis must be cleared in order to avoid recurrence of thrombus. Typically, the venous stenosis is treated with balloon angioplasty after the graft has been cleared of thrombus. Balloon angioplasty is expensive, time-consuming and often is not successfully in totally clearing the obstruction due to the very high pressures required to expand the stenosis. Cutting wire balloons must sometimes be used to successfully restore normal blood flow.

Graft infections often occur in thrombosed grafts. Current treatment options include prolonged administration of antibiotic and antimicrobial drugs and surgical intervention. Pharmacological solutions are slow acting and often take days before improvement is shown. Resistant infectious strains may reduce the probability of the infection clearing. As a result of these problems, surgical treatment is considered the gold standard for treating infected grafts. Surgery often involves explantation of the graft and debridement of infected tissue. The graft is either removed and replaced, or reattached at a non-infected area. Along with the high costs and complication rates of surgery, this option removes the graft as a viable access route for at least two to four weeks.

All of the current options for treating graft complications adversely affect a patient's dialysis schedule, cause patient discomfort, and may result in temporary or permanent loss of the original access site. Therefore, it is desirable to provide a device and method for the treatment of graft complications including thrombosis, infection and stenosis with a safe, easy, and reliable manner without the need for pharmacological treatments and/or surgical intervention.

### SUMMARY OF THE DISCLOSURE

Throughout the present teachings, any and all of the one, two, or more features and/or components disclosed or suggested herein, explicitly or implicitly, may be practiced and/or implemented in any combinations of two, three, or more thereof, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the claimed inventions presented herein or in any future applications claiming priority to the instant application.

Disclosed herein are devices and methods for delivering electrical pulses for treatment of a complication, such as thrombosis, stenotic segments, or infections, associated with an arteriovenous graft or fistula. In particular, according to one embodiment of the present invention, a method includes positioning at least two electrodes near or within a target zone of complication formed on the graft or fistula; and applying between the positioned electrodes electrical pulses in an amount sufficient to subject substantially all cells within the target zone to electroporation. In one embodiment, the method is carried out by delivering electrical pulses in an amount sufficient to subject substantially all cells within the target zone to irreversible electroporation without creating a thermally damaging effect. In one embodiment, the at least two electrodes are carried on a balloon catheter which is adapted to be removably positioned inside an arteriovenous graft and near the treatment zone. In another embodiment, a pair of electroporation probes are positioned near the graft and surrounds the treatment area, wherein each probe carries one of the at least two electrodes. In another embodiment, a single probe carries the at least two electrodes.

### BRIEF DESCRIPTION OF THE DRAWINGS

FIG. 1 is a cross-sectional view of an arteriovenous graft connecting an artery and vein and illustrating areas of stenotic build-up.

FIG. 2 illustrates an electroporation balloon catheter with a plurality of electrodes at the distal segment.

FIG. 3A is an enlarged view of the distal segment of the electroporation balloon catheter of FIG. 2 with the balloon in a collapsed position.

FIG. 3B is an enlarged view of the distal segment of the electroporation balloon catheter of FIG. 2 with the balloon in an expanded position.

FIG. 4A is a cross-sectional view of an arteriovenous graft connecting an artery and vein with the electroporation balloon catheter positioned through the graft and into the vein.

FIG. 4B is a cross-sectional view of an arteriovenous graft connecting an artery and vein with the electroporation balloon catheter being shown with the balloon expanded against a stenotic segment of the vein.

FIG. 5 is an enlarged cross-sectional view of the balloon within the stenotic segment of the vein taken along lines 5-5 of FIG. 4B.

FIG. 6A is a cross-sectional view of an arteriovenous graft connecting an artery and vein with the electroporation balloon catheter being shown with the collapsed balloon positioned against a stenotic segment of the arteriovenous graft.

FIG. 6B is a cross-sectional view of an arteriovenous graft connecting an artery and a vein with the electroporation balloon catheter being shown with the balloon expanded against a stenotic segment of the arteriovenous graft.

FIG. 7 is a cross-sectional view of an arteriovenous graft connecting an artery and a vein following treatment with the electroporation balloon catheter.

FIG. 8 illustrates an electroporation probe according to another embodiment of the present invention.

FIGS. 9-10 illustrate cross-sectional views of an arteriovenous graft connecting an artery and a vein showing the electrical field gradient surrounding a pair of electroporation probes which have been inserted and positioned adjacent to stenotic areas of the vein and graft.

FIG. 11 illustrates a flow chart depicting a first embodiment of the method of the present invention

FIG. 12 illustrates a flow chart depicting a second embodiment of the method of the present invention.

### DETAILED DESCRIPTION OF THE INVENTION

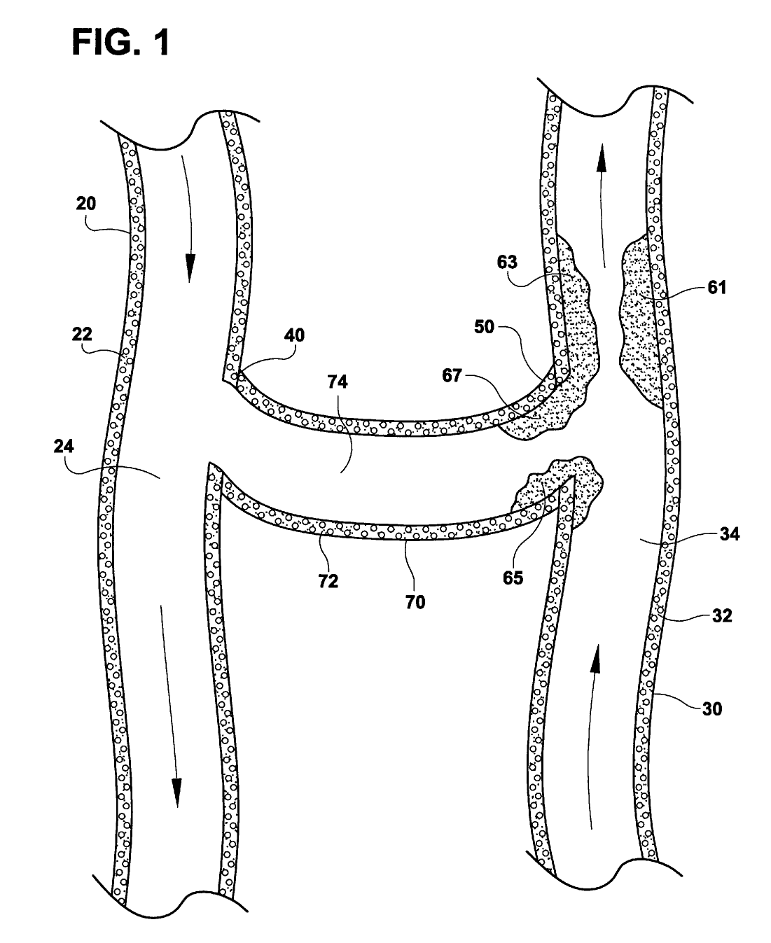

The present invention can be understood by reference to FIGS. 1 through 12. FIG. 1 illustrates an arteriovenous (AV) graft connecting an artery and vein and illustrating areas of stenotic build-up. As mentioned above, as used herein, “graft” is inclusive of an A/V graft and A/V fistula. Artery 20 has an arterial wall 22 and lumen 24. Blood flows away from the heart through artery 20 in the direction of the arrows. Vein 32 has a vein wall 30 and lumen 34 through which blood flows at a lower pressure toward the heart in the direction of the arrows. Connecting vein 32 to the artery 20 is AV graft 70 with graft wall 72 and graft lumen 74. Graft 70 is surgically connected to artery 20 at arterial anastomosis 40 and to vein 32 at venous anastomosis 50. A portion of the blood volume flowing through artery lumen 24 will be diverted through graft lumen 74. The graft blood flows through the graft lumen 74, entering the vein blood flow through venous anastomosis 50. During dialysis procedures, blood is removed via a first needle (not shown) through the graft wall 72. Cleansed blood is returned through a second needle (not shown) placed in the graft 70 downstream of the first needle.

Various complications can develop with arteriovenous grafts or fistulas, such as thrombosis, stenotic segments, or infections. Although the following discussion focuses on the treatment of stenotic segments, it should be understood that the present invention can be used to treat any one of these types of complications by treating any target zone of complication.

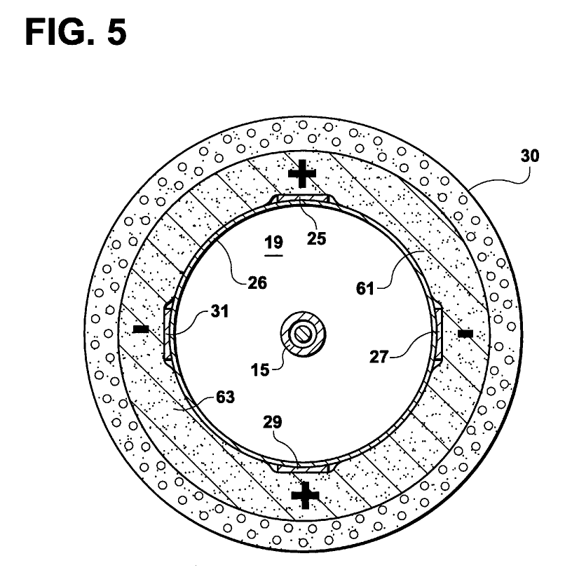

FIG. 1 illustrates stenotic segments 61, 63, 65 and 67. These stenotic segments represent hyperplastic neointimal thickening of the vessel wall which commonly occurs at both the venous anastomosis 50 and arterial anastomosis 40, as well as in the native vein segment adjacent to the venous anastomosis 50. The narrowing of the graft lumen 74 and the vein lumen 34 at stenotic segments 61, 63, 65 and 67 causes a slow down or obstruction of blood flow resulting in the formation of thrombus within the graft 70. The stenotic segments are illustrated as separate segments in the figures only because the views are cross-sectional. It should be understood that in some sections, the stenotic segments can collectively form a continuous annular segment which resides along the entire circumference of the inner vessel wall, as shown in FIG. 5.

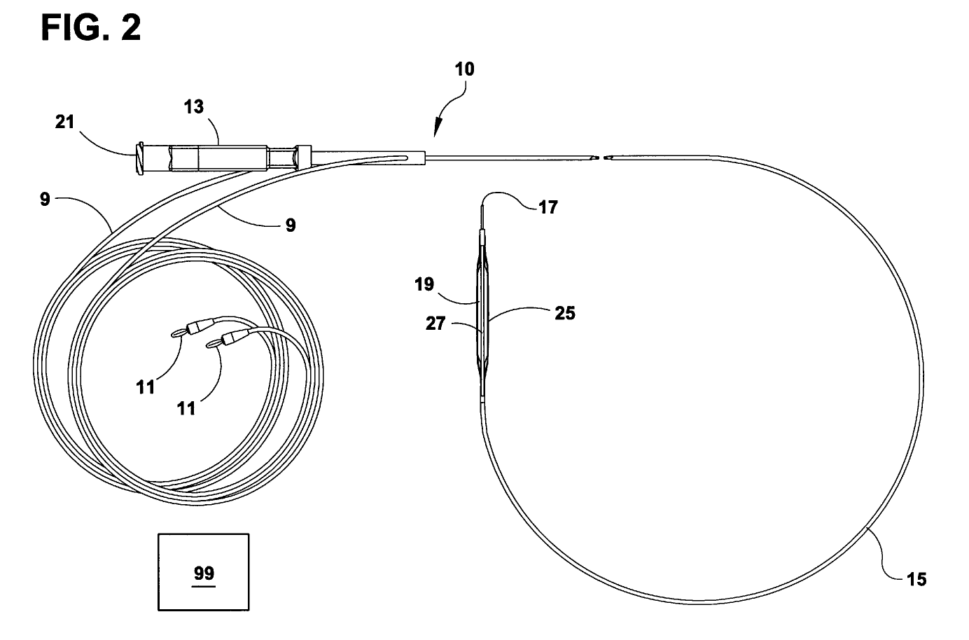

FIG. 2 shows one embodiment of an electroporation device or catheter 10 capable of treating stenotic segments associated with a vascular graft. Although the following discussion relates to the treatment of stenotic segments, it should be understood that the same applies to the treatment of thrombosis and/or infection in the same manner. The catheter 10 is comprised of hub 13, a flexible catheter shaft 15 extending distally from hub 13 to an expandable member such as a balloon 19 and terminating in distal tip 17. Hub 13 includes a port opening 21 in communication with a shaft lumen (not shown) for guidewire tracking or injection and aspiration of fluids to expand or collapse the balloon 19 during use. Shaft 15 extends from hub 13 distally through the interior or the balloon 19. Balloon 19 is coaxially arranged around catheter shaft 15 near the distal end and is shown in an expanded state. Although not shown in FIG. 2, catheter 10 may include a side arm extension on hub 13 for inflation and deflation of the balloon 19. Extending from hub 13 are electrical cable wires 9 which terminate in connectors 11. Connectors 11 are connected to an electrical pulse generator 99 to provide an electrical current to a plurality of longitudinal electrodes 25 and 27, which are positioned in a longitudinal arrangement around the surface of balloon 19.

The size and shape of the electrodes 25, 27 can vary. For example, the electrodes can be ring-shaped, spiral-shaped (helical configuration), or can exist as segmented portions. The electrodes may also be a series of strips placed longitudinally along the balloon surface. The electrodes may be comprised of any suitable electrically conductive material including but not limited to stainless steel, gold, silver and other metals. Other embodiments for the configuration of the balloon and electrodes can include those described in U.S. application Ser. No. 12/413,357, filed Mar. 27, 2009, entitled “BALLOON CATHETER METHOD FOR REDUCING RESTENOSIS VIA IRREVERSIBLE ELECTROPORATION”, which is fully incorporated by reference herein.

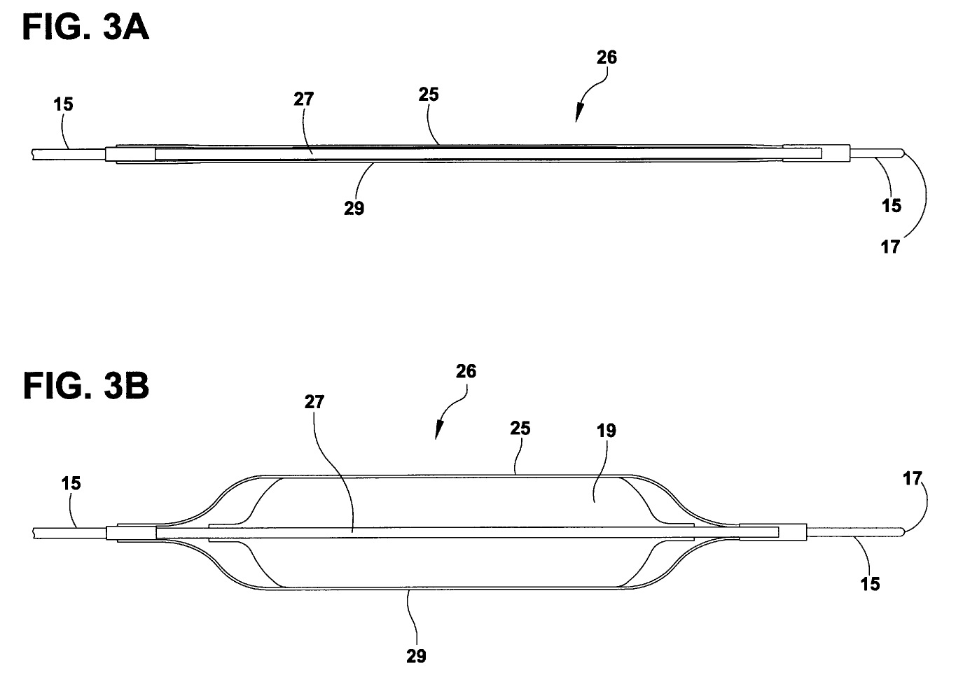

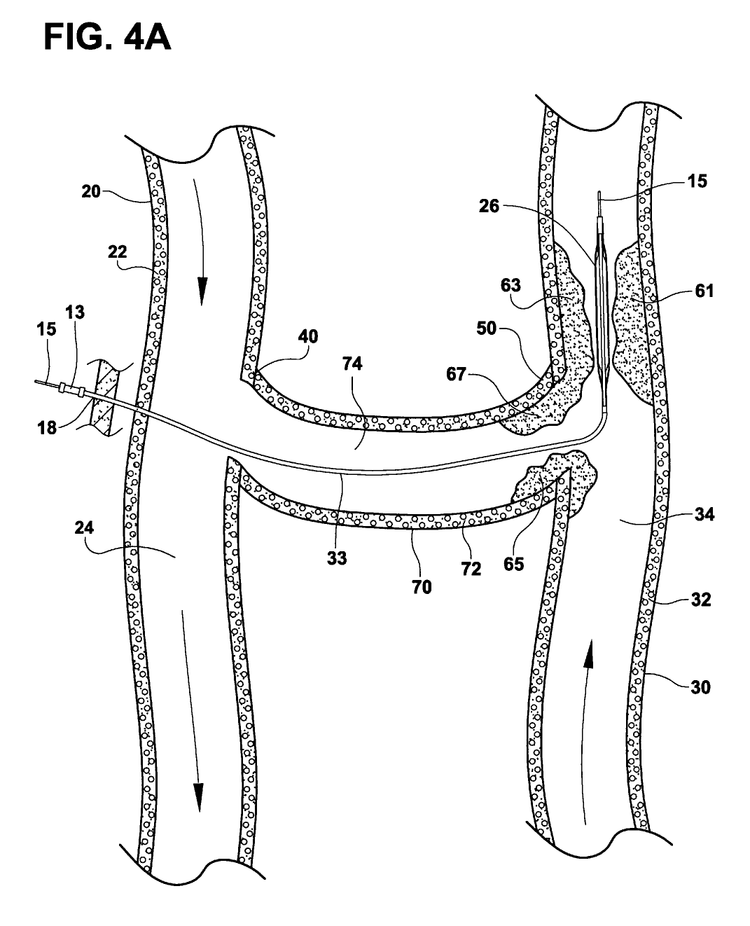

FIGS. 3A and 3B illustrate enlarged views of the distal segment of the electroporation balloon catheter 10 of FIG. 2 with the balloon in a deflated position (FIG. 3A) and an inflated position (FIG. 3B). Balloon 19 (not visible in FIG. 3A) is coaxially arranged around catheter shaft 15 near the distal end of the shaft. Also illustrated is electrode assembly 26, which is comprised of a plurality of longitudinal electrodes 25, 27, 29 and 31 (electrode 31 is hidden behind the balloon 19; see FIG. 5). The plurality of longitudinal electrodes form a cage which expands when the balloon 19 is inflated, as shown in FIG. 3B. The plurality of longitudinal electrodes, when energized, create an electrical current which irreversibly electroporates the stenotic regions of the vessels and/or graft, as will be explained in greater detail below.

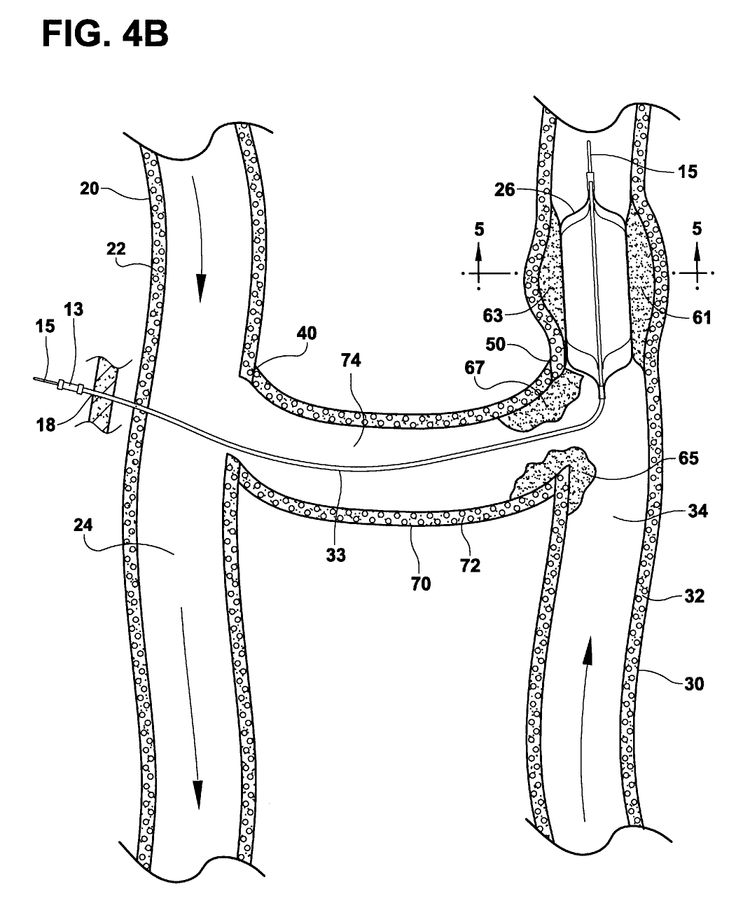

To treat the stenotic regions of the arteriovenous graft and connecting vessels, the electroporation catheter is introduced into the graft, as shown in FIG. 4A. Using standard techniques known in the art, the graft is accessed using a needle and guidewire. The electroporation catheter is then inserted over the wire into the graft 70 through a skin insertion site 18. The catheter is advanced through the graft 70 into the vein lumen 34. The balloon 19 of the catheter is positioned within the stenotic segments 61 and 63. In one embodiment, the balloon 19 is positioned with the help of imaging devices as are known in the art.

As shown in FIG. 4B, the balloon 19 is then inflated which causes the electrode assembly 26 to expand outward and contact the inner vessel wall at the stenotic segments 61 and 63. The pressure from the expanded balloon 19 forces the stenotic segments 61 and 63 to be pushed radially outward restoring the original luminal diameter of the vein 32. This process is commonly known in the art as balloon angioplasty.

However, certain side effects and complications can result from the angioplasty procedure. Angioplasty triggers the proliferation of smooth muscle cell growth on the inner wall of the treated vessel. When the stenotic segments are pushed radially outward by the pressure of the expanded balloon 19, cracks occur in the stenotic segments causing vessel wall damage, also known as barotrauma. In an attempt to repair itself, the vessel wall responds to barotrauma by triggering the rapid growth of smooth muscle cells along the inner lining of the treated vessel segment. This causes a thickening of the overall vessel wall and consequently, a reduction in the luminal diameter of the vessel as shown in FIG. 1.

The present invention helps to mitigate these complications. In one aspect of the current invention, a method of treating the vein segment and/or graft segment uses the above described angioplasty procedure in combination with electrical currents to irreversibly electroporate the treated vessel segment and/or graft segment, thereby suppressing the proliferation of smooth muscle cell growth.

Electroporation is defined as a phenomenon that makes cell membranes permeable by exposing them to certain electric pulses. As a function of the electrical parameters, electroporation pulses can have two different effects on the permeability of the cell membrane. The permeabilization of the cell membrane can be reversible or irreversible as a function of the electrical parameters used. Reversible electroporation is the process by which the cellular membranes are made temporarily permeable. The cell membrane will reseal a certain time after the pulses cease, and the cell will survive. Reversible electroporation is most commonly used for the introduction of therapeutic or genetic material into the cell. Irreversible electroporation, also creates pores in the cell membrane but these pores do not reseal, resulting in cell death.

Irreversible electroporation has recently been discovered as a viable alternative for the ablation of undesired tissue. See, in particular, PCT Application No. PCT/US04/43477, filed Dec. 21, 2004. An important advantage of irreversible electroporation, as described in the above reference application, is that the undesired tissue is destroyed without creating a thermally damaging effect. When tissue is ablated with thermally damaging effects, not only are the cells destroyed, but the connective structure (tissue scaffold) and the structure of blood vessels are also destroyed, and the proteins are denatured. This thermal mode of damage detrimentally affects the tissue, that is, it destroys the vasculature structure and bile ducts, and produces collateral damage.

Irreversible and reversible electroporation without thermally damaging effects to ablate tissue offers many advantages. One advantage is that it does not result in thermal damage to target tissue or other tissue surrounding the target tissue, and therefore does not damage blood vessels. Another advantage is that it only ablates living cells and does not damage non-cellular or non-living materials such as implanted medical devices (arteriovenous grafts for example).

The irreversible electroporation treatment according to the present invention may be carried out prior to, during or after the angioplasty procedure. Alternatively, the irreversible electroporation treatment may be carried out in lieu of angioplasty. Irreversible electroporation suppresses the proliferation response of the vessel by selectively destroying the smooth muscle cells. Since irreversible electroporation may be non-thermal treatment modality within specific parameters, the vessel and adjacent structures are not damaged by the electrical field. As an example, the connective non-cellular tissue of the vessel which consists of collagen, elastin and other extra-cellular components is not affected by the non-thermal electrical current. Instead, the treated vessel wall is gradually repopulated with endothelial cells that regenerate over a period of time but will not thicken into a stenotic lesion.

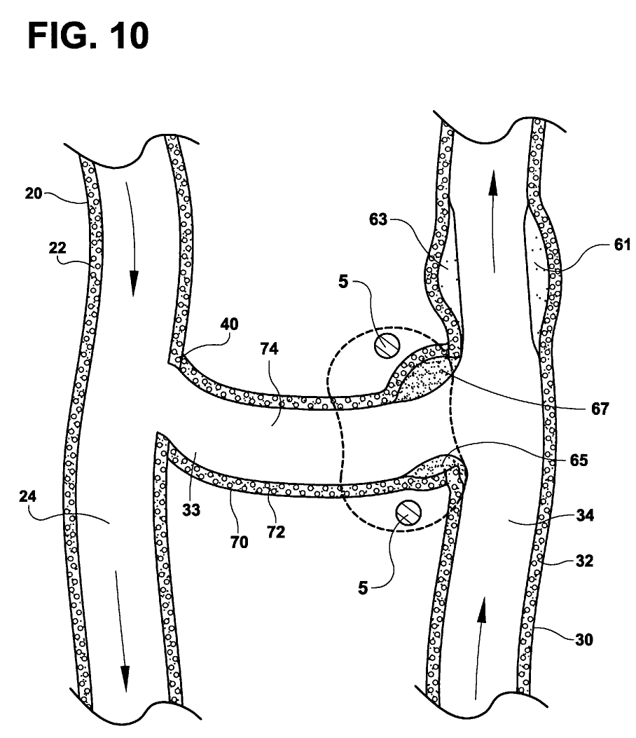

The electrodes 25, 27, 29 and 31 are adapted to administer electrical pulses as necessary in order to reversibly or irreversibly electroporate the cell membranes of the cells comprising the stenotic segments 61, 63, 65, 67 located near the arteriovenous graft. By varying parameters of voltage, number of electrical pulse and pulse duration, the electrical field will either produce irreversible or reversible electroporation of the cells within the treatment zone. Typical ranges include but are not limited to a voltage level of between 50-8000 Volts/cm, a pulse duration of between 5-500 microseconds, and between 2-500 total pulses. The electroporation treatment zone is defined by mapping the electrical field that is created by the electrical pulses between two electrodes (see, for example, the dashed lines surrounding the electrodes 5, 5 in FIGS. 9-10 which represent the boundary line of the treatment zone). The actual ranges used will depend on the tissue type as well as other factors. Preferred ranges for irreversible electroporation include a voltage level of between 500-1500 Volts/cm, a pulse duration of between 50-150 microseconds, and between 40-150 total pulses. Preferably, the pulses are delivered in sets with at least a one second delay between sets. For example, 9 sets of 10 pulses per set can be delivered.

When electrical pulses are administered within the irreversible parameter ranges, permanent pore formation occurs in the cellular membrane, resulting in cell death of the smooth muscle cells of the stenotic segments. In another aspect, by proactively administering the electrical pulses according to a predetermined schedule, stenotic growths near the arteriovenous graft 70 can be prevented altogether. Application of electrical pulses applied to the arteriovenous graft 70 at regular intervals post-implantation may be effective in preventing thrombosis, stenotic growths and/or infections.

Alternatively, electrical pulses may be administered within a reversible electroporation range in combination with drugs to treat thrombosis, stenotic growths and/or infections associated with the arteriovenous graft. The ranges for creating reversible electroporation will depend on tissue type as well as other factors. See, for example, US Patent Application Publication No. 2007/0043345 to Devalos et al., which is incorporated by reference herein. The effectiveness of therapeutic agents may be enhanced through reversible electroporation by temporarily opening pores in the target cells within the clot to allow the uptake of drug within the cell. In another aspect of the invention, anti-infective drugs such as antibacterial, anti-viral and anti-fungal agents may be delivered concurrently with the electrical pulses in either irreversible or reversible ranges to increase the impact of the therapeutic agent on the target complication.

FIG. 5 is a cross-sectional view of the stenotic vein segment taken along lines 5-5 of FIG. 4B. Vein wall 30 with stenotic segments 61, 63 (collectively forming an annular segment) coaxially surrounds the inflated balloon 19. Positioned between the outer surface of the inflated balloon and the stenotic region are longitudinal electrodes 25, 27, 29, and 31. The electrical current flow pattern is shown in FIG. 5. Electrical energy will be transmitted from an electrical generator to longitudinal electrodes 25, 27, 29, and 31 of the electrode assembly. In one embodiment, a first electrical pathway is of a positive polarity as indicated by the “+” signs in FIG. 5. Electrical energy of a positive polarity may be transmitted through a wire to the electrodes 25 and 29 of the longitudinal electrode assembly. In one embodiment, a second electrical pathway is of a negative polarity as indicated by the “−” signs in FIG. 5. Electrical energy of a negative polarity may be transmitted through a wire to the electrodes 27 and 29 of the longitudinal electrode assembly.

The electrodes can be electrically energized one pair at a time and selectively switched to cover all four pairs. In the embodiment shown, all electrodes are simultaneously energized, causing electrical current to flow between positive polarity electrodes 25, 29 and negative polarity electrodes 27, 31. As an example, electrical current will flow from electrode 27 with a negative polarity to electrodes 25 and 29 with a positive polarity. In a similar manner, electrical current will flow from negative polarity electrode 31 to both positive polarity electrodes 25 and 29. The electric field (target zone) established by the applied current should be sufficiently large to cover all of the target stenotic segments to be ablated.

Although not shown in FIG. 5, the flow of electrical current will be restricted to the un-insulated (exposed) portions of the electrodes, which correspond with maximum diameter of the inflated balloon 19. The resulting combined electrical fields created by the application of electrical energy of opposite polarities to the electrodes 25, 27, 29, 31 create a substantially 360 degree electrical field target zone surrounding the balloon 19. When the catheter is in position in a target vessel, this combined electrical field zone extends radially outward and into the inner wall of the vessel. In this manner, the entire circumference of the inner wall of the target vessel is subject to a therapeutic electrical field.

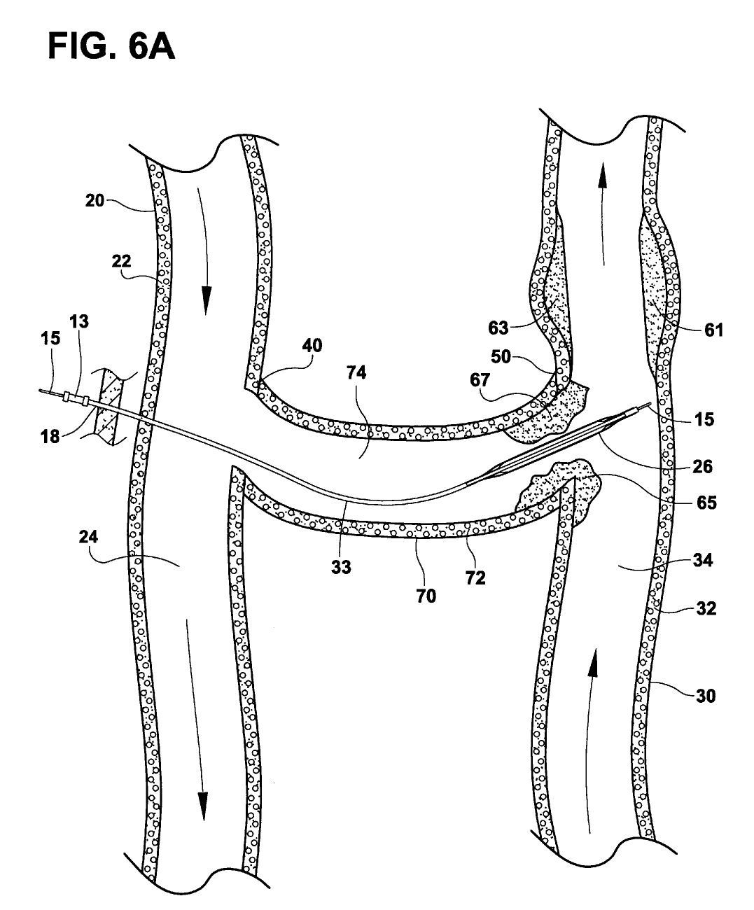

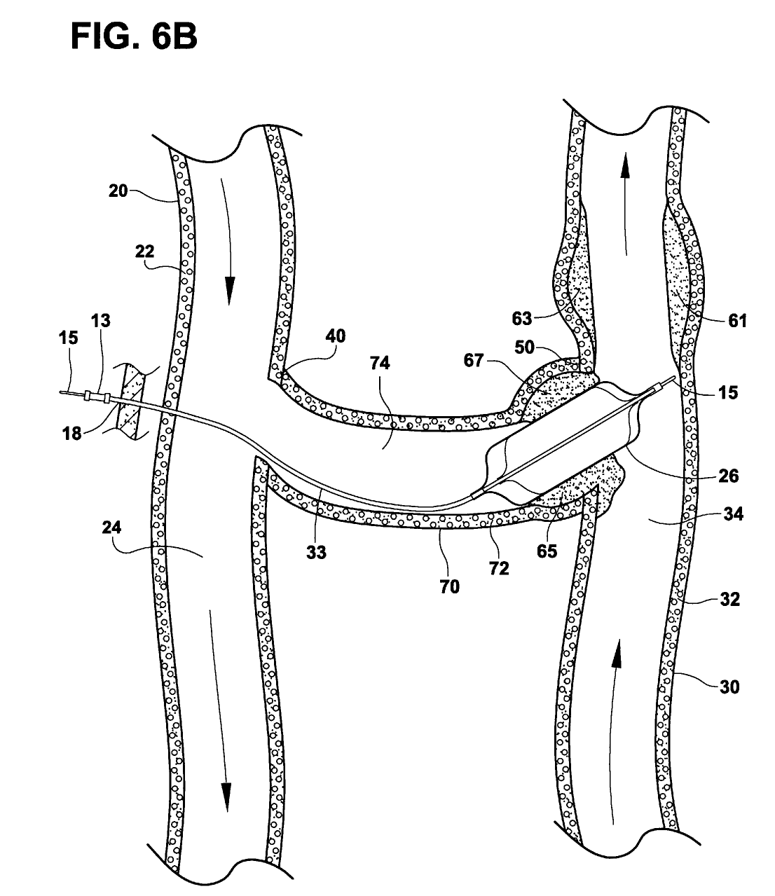

Turning now to FIG. 6A, once the stenotic segments 63 and 61 have been treated with angioplasty and irreversible electroporation, the balloon catheter is then repositioned within the graft 70 at the venous anastomosis 50. The balloon is then inflated to push the stenotic segments 65 and 67 outwardly as shown in FIG. 6B. Once the original luminal diameter of the graft 70, near the venous anastomosis 50, has been reestablished, electrical currents are applied to the treated area to suppress any smooth muscle cell growth.

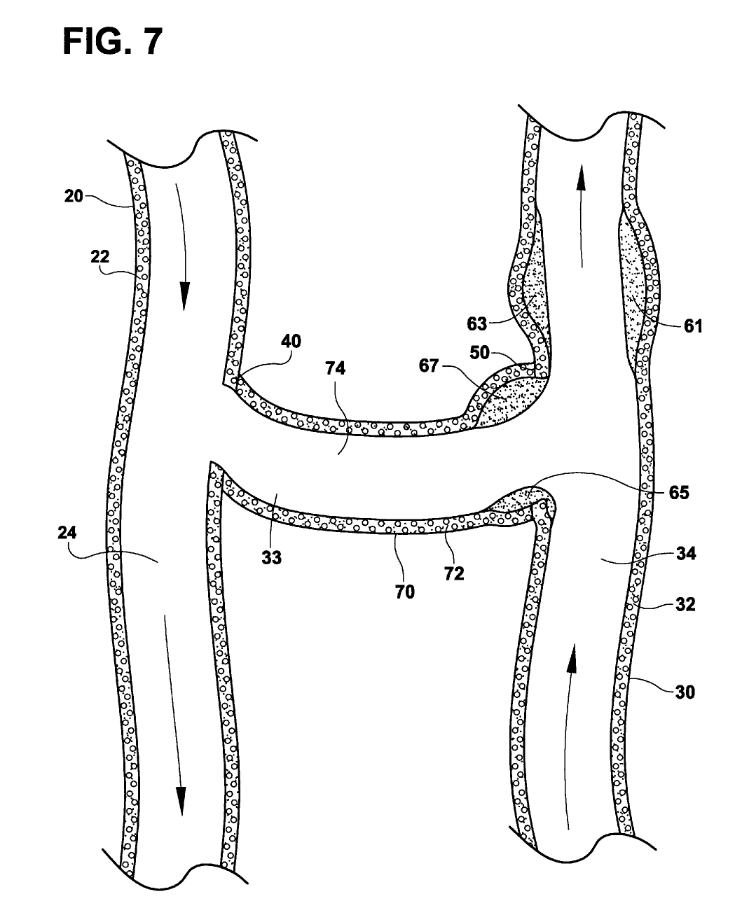

FIG. 7 illustrates the graft 70 and vein 32 after treatment has been completed.

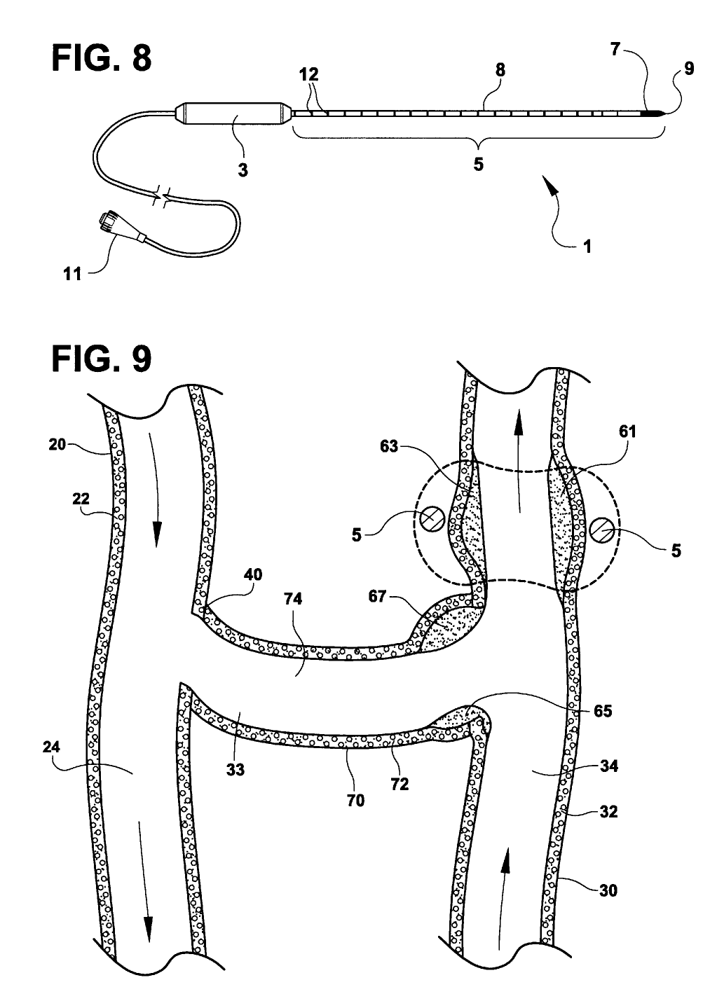

FIG. 8 illustrates an electroporation probe that can be used with the present invention. The probe 1 includes an electrical connector 11 that is adapted to be connected to a generator. An electrode 5 extends distally from a handle 3. The electrode includes a plurality of depth indicators 12 along an insulated segment of the shaft. The electrode includes a distal active portion 7 and terminates at a sharp distal tip 9. The distal tip 9 is adapted to pierce tissue. The probe 1 is designed with a mechanism to slide the insulation sleeve 8 to selectively change the exposed distally active portion to cover varying target zone sizes. In another embodiment, the single electroporation probe can be a bipolar probe which includes at least two electrodes. This single electroporation probe can be used to treat areas of complication as defined herein. In one embodiment, the probe can be a catheter with at least two electrodes disposed on an outer wall of the catheter which is used to treat a target zone, especially if no angioplasty is required.

FIGS. 9-10 illustrate cross-sectional views of an arteriovenous graft connecting an artery and a vein showing the electrical field gradient surrounding two electroporation probes which have been inserted and positioned adjacent to stenotic areas of the vein and graft. As shown in FIG. 9, a pair of electrodes 5, 5 can be inserted and positioned so as to surround stenotic segments 61 and 63. As shown in FIG. 10, a pair of electrodes can also be inserted and positioned so as to surround stenotic segments 65 and 67. When using a pair of electrodes to treat the tissue, one electrode has a positive polarity and the other electrode has a negative polarity so as to generate an electroporation field to irreversibly electroporate the treated vessel segment, thereby suppressing the proliferation of smooth muscle cell growth. In other embodiments, a single bi-polar probe or any number of probes greater than two can be used. Although FIGS. 9-10 illustrate the condition of the stenotic segments after the angioplasty procedure has been used as described above, the electroporation probes 1 can be used to irreversibly electroporate the tissue prior to, during or after the angioplasty procedure. Alternatively, the irreversible electroporation treatment may be carried out in lieu of angioplasty. Irreversible electroporation suppresses the proliferation response of the vessel by selectively destroying the smooth muscle cells as described above. In another aspect, by proactively administering the electrical pulses according to a predetermined schedule, stenotic growths near the arteriovenous graft 70 can be prevented altogether. Application of electrical pulses applied to the arteriovenous graft 70 at regular intervals post-implantation may be effective in preventing thrombosis, stenotic growths and/or infections.

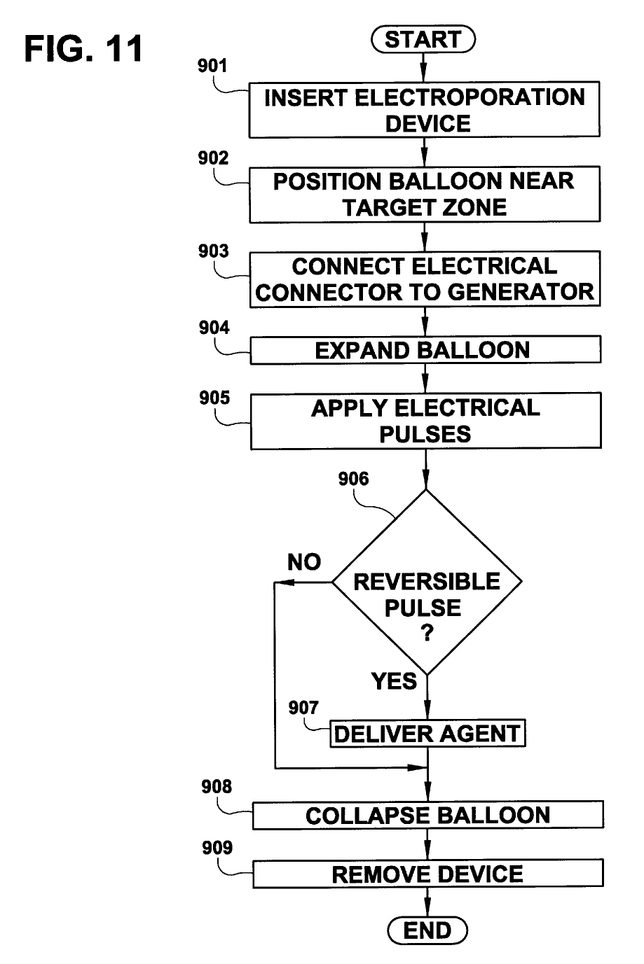

Referring now to FIG. 11, the method of performing electroporation treatment using the electroporation device 10 depicted in FIG. 2 is illustrated. After the thrombosis, stenotic segments, and/or infection has been detected and the location of the formation determined using ultrasound or fluoroscopic imaging, electroporation device 10 (FIG. 2) is inserted into the arteriovenous graft 70 (901). The balloon 19 is then positioned relative to the stenotic segment as previously described (902). The electrical connectors 11 are then connected to an electrical generator (903). The balloon is expanded (see FIG. 3B) (903). Electrical pulses are then applied across the electrodes (905) creating a field gradient sufficient to non-thermally electroporate the cells present in the thrombosis, stenotic segments, and/or infections. If the electrical generator treatment parameters are set to deliver electrical pulses within the reversible range (906), therapeutic agents may be delivered through the catheter lumen (907) passing into the thrombosis, stenotic segments, and/or infections through either side holes or end holes of the catheter. Alternatively, the electroporation device may be configured to include a lumen through which agents may be administered. The therapeutic agents can be delivered prior to, during, or after the delivery of electrical pulses. If there are multiple stenotic segments 61, 63, 65, 67 (see FIG. 1), then they are treated individually to the extent necessary. The balloon is then collapsed (908). After the procedure is complete, the electroporation device is removed from the arteriovenous graft (909). Non-thermal amelioration of the thrombosis, stenotic segments, and/or infections occur after electroporation treatment.

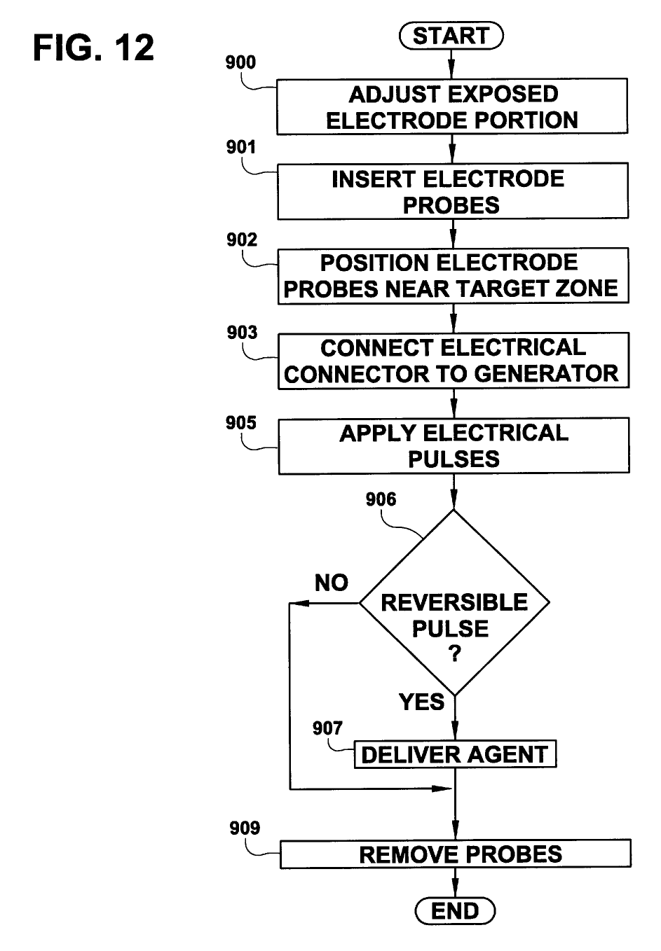

Referring now to FIG. 12, the method of performing electroporation treatment using the electroporation probe 1 depicted in FIG. 8 is illustrated. After the thrombosis, stenotic segments, and/or infection has been detected and the location of the formation determined using ultrasound or fluoroscopic imaging, the amount of exposed portion 7 is adjusted by sliding the insulating sleeve (900). Then, a pair of electroporation probes 1 (FIG. 8) are inserted near the arteriovenous graft 70 (901). The distal active portions 7 of each probe is then positioned relative to the stenotic segment as previously described (902). The electrical connectors 11 are then connected to an electrical generator (903). Electrical pulses are then applied across the electrodes (905) creating a field gradient sufficient to non-thermally electroporate the cells present in the thrombosis, stenotic segments, and/or infections. If the electrical generator treatment parameters are set to deliver electrical pulses within the reversible range (906), therapeutic agents may be delivered through the catheter lumen (907) passing into the thrombosis, stenotic segments, and/or infections through either side holes or end holes of the catheter. Alternatively, the electroporation device may be configured to include a lumen through which agents may be administered. The therapeutic agents can be delivered prior to, during, or after the delivery of electrical pulses. If there are multiple stenotic segments 61, 63, 65, 67 (see FIG. 1), then they are treated individually to the extent necessary. After the procedure is complete, the electroporation probes are removed from the patient (909). Non-thermal amelioration of the thrombosis, stenotic segments, and/or infections occur after electroporation treatment.

The present invention affords several advantages. Thrombosis, stenotic segments and/or infections are destroyed without having to remove the arteriovenous graft from the patient. The treatment is minimally-invasive and highly efficacious. Because irreversible electroporation does not create thermal activity, the arteriovenous graft is not damaged by the treatment. Thrombosis, stenotic segments, and/or infections are treated quickly, and the arteriovenous graft can be maintained according to a predetermined schedule to insure that the lumens of the graft and connected blood vessels remain clear.

In other embodiments, this invention can be used to treat any area of complication in any other non-vascular tubular structures in the body, such as stenotic regions associated with a bile duct, or infections or lesions associated with the esophagus (i.e. esophageal cancer).

The above disclosure is intended to be illustrative and not exhaustive. This description will suggest many modifications, variations, and alternatives may be made by ordinary skill in this art without departing from the scope of the invention. Those familiar with the art may recognize other equivalents to the specific embodiments described herein. Accordingly, the scope of the invention is not limited to the foregoing specification.

What is claimed is:

1. A method for the treatment of a complication associated with an arteriovenous graft or fistula comprising:
positioning at least two electrodes near a target zone of complication formed on the graft or fistula; and
applying between the positioned electrodes electrical pulses in an amount sufficient to subject substantially all cells within the target zone to irreversible electroporation.

2. The method of claim 1, further comprising inserting into the arteriovenous graft or fistula a balloon catheter, a balloon of the balloon catheter carrying the at least two electrodes.

3. The method of claim 2, wherein the step of positioning includes:
positioning the balloon near the target zone; and
expanding the balloon so as to position the at least two electrodes.

4. The method of claim 1, wherein the step of positioning includes:
positioning at least a pair of electroporation probes near the target zone and outside of the arteriovenous graft or fistula, each probe carrying at least one electrode.

5. The method of claim 4, further comprising:
for each probe, adjusting the amount of exposed electrode in relation to the size of the target zone.

6. The method of claim 1, wherein the step of positioning includes:
positioning an electroporation probe near the target zone, the probe carrying the at least two electrodes.

7. The method of claim 1, further comprising:
inserting into the arteriovenous graft or fistula a catheter having the at least two electrodes disposed on an outer wall of the catheter.

8. The method of claim 1, wherein the step of applying includes applying electrical pulses whose pulse amplitude is in a range of 500 Volt/cm and 1500 Volt/cm.

9. The method of claim 1, wherein the step of applying includes applying electrical pulses whose duration is in a range of 50 microseconds and 150 microseconds.

10. The method of claim 1, wherein the step of applying includes applying electrical pulses whose amplitude is in the range of 500 Volt/cm and 1500 Volt/cm and whose duration is in a range of 50 microseconds and 150 microseconds.

11. A method for the treatment of a complication associated with an arteriovenous graft or fistula comprising:
positioning at least two electrodes near a target zone of complication formed on the graft or fistula;
connecting a voltage generator to the at least two electrodes;
applying between the positioned electrodes electrical pulses from the voltage generator in an amount sufficient to subject substantially all cells within the target zone to irreversible electroporation without creating a thermally damaging effect to a majority of the tissue within the target zone.

12. The method of claim 11, further comprising inserting into the arteriovenous graft or fistula a balloon catheter, a balloon of the balloon catheter carrying the at least two electrodes.

13. The method of claim 12, wherein the step of positioning includes:
positioning the balloon near the target zone; and
expanding the balloon so as to position the at least two electrodes.

14. The method of claim 11, wherein the step of positioning includes:
positioning at least a pair of electroporation probes near the target zone and outside of the arteriovenous graft or fistula, each probe carrying at least one electrode.

15. The method of claim 14, further comprising:
for each probe, adjusting the amount of exposed electrode in relation to the size of the target zone.

16. The method of claim 11, wherein the step of positioning includes:
positioning an electroporation probe near the target zone, the probe carrying the at least two electrodes.

17. The method of claim 11, further comprising:
inserting into the arteriovenous graft or fistula a catheter having the at least two electrodes disposed on an outer wall of the catheter.

18. The method of claim 11, wherein the step of applying includes applying electrical pulses whose pulse amplitude is in a range of 500 Volt/cm and 1500 Volt/cm.

19. The method of claim 11, wherein the step of applying includes applying electrical pulses whose duration is in a range of 50 microseconds and 150 microseconds.

20. The method of claim 11, wherein the step of applying includes applying electrical pulses whose amplitude is in the range of 500 Volt/cm and 1500 Volt/cm and whose duration is in a range of 50 microseconds and 150 microseconds.

21. A method for the treatment of a complication associated with an arteriovenous graft or fistula comprising:
inserting into the arteriovenous graft or fistula a balloon catheter whose balloon carries at least two electrodes;
positioning the at least two electrodes near a target zone of complication formed on the graft or fistula;
connecting the at least two electrodes to a voltage generator;
expanding the balloon;
applying between the connected electrodes electrical pulses in an amount sufficient to subject substantially all cells within the target zone to irreversible electroporation without creating a thermally damaging effect;
collapsing the balloon;
removing the balloon catheter from the arteriovenous graft or fistula.

22. The method of claim 21, wherein the step of expanding includes injecting fluid into the balloon, and wherein the step of collapsing includes removing fluid from the balloon.

23. A method for the treatment of a complication associated with an arteriovenous graft or fistula comprising:
positioning at least two electrodes near a target zone of complication formed on the graft or fistula;
delivering a therapeutic agent to the target zone of complication;
applying between the positioned electrodes electrical pulses in an amount sufficient to subject substantially all cells within the target zone to reversible electroporation.

24. The method of claim 23, further comprising inserting into the arteriovenous graft or fistula a balloon catheter, a balloon of the balloon catheter carrying the at least two electrodes.

25. The method of claim 23, wherein the step of positioning includes:
positioning at least a pair of electroporation probes near the target zone and outside of the arteriovenous graft or fistula, each probe carrying at least one electrode.

26. The method of claim 25, further comprising:
for each probe, adjusting the amount of exposed electrode in relation to the size of the target zone.

27. The method of claim 23, further comprising:
inserting into the arteriovenous graft or fistula a catheter having the at least two electrodes disposed on an outer wall of the catheter.
